(19) United States
(12) Patent Application Publication
(10) Pub. No.: US 2023/0274528 A1
(43) Pub. Date: Aug. 31, 2023

(54) SYSTEM AND METHOD FOR ASSISTING WITH THE DIAGNOSIS OF OTOLARYNGOLOGIC DISEASES FROM THE ANALYSIS OF IMAGES

(71) Applicant: UNIVERSIDAD TÉCNICA FEDERICO SANTA MARIA, Viña del Mar (CL)

(72) Inventors: Michelle VISCAINO, Valparaíso (CL); Fernando AUAT CHEEIN, Viña del Mar (CL)

(21) Appl. No.: 18/016,322

(22) PCT Filed: Jul. 15, 2020
(86) PCT No.: PCT/IB20/56636

§ 371 Date: Jan. 13, 2023

(51) Int. Cl. G06V 10/764; G06T 7/00; G06V 10/82; G16H 30/40

(52) U.S. Cl. CPC G06V 10/764; G06T 7/0012; G06V 10/82; G16H 30/40; G06T 2200/24; G06T 2207/10068; G06T 2207/20084; G06T 2207/30168; G06T 2207/20061; G06T 2207/20081; G06T 2207/30004; G06V 2201/03

(57) ABSTRACT

The present invention provides a system and a method for assisting in the diagnosis of diseases from otolaryngology images that comprises: an apparatus for the acquisition of images of otolaryngologic endoscopy; a processor, operatively connected to said apparatus for the acquisition of otolaryngologic endoscopy images; and a user interface comprising a screen, said user interface operatively connected to said processor; wherein said processor is configured to: recognize a type of otolaryngologic endoscopic examination apparatus to which said apparatus belongs; obtain a plurality of images of otolaryngologic endoscopy from said apparatus; display said plurality of images on said screen; and identify, from said plurality of images, whether the same corresponds to any disease or to a healthy patient.

### TECHNICAL FIELD OF THE INVENTION

The present invention relates to the field of medical technologies, more specifically to the diagnosis and identification field and in particular it provides an ex vivo system and method for assisting with the diagnosis of diseases from otolaryngology images of an area under examination.

### BACKGROUND OF THE INVENTION

The diagnosis of otolaryngologic diseases, mainly those related to the ear, nose, and throat is commonly carried out through a medical appointment and the physical exam of the area under examination. The subjective nature of this procedure results in the clinical diagnosis being affected by the bias introduced by the observer and his experience and diagnostic skills.

For example, studies conducted in both specialist and non-specialist physicians have shown diagnostic accuracy in ear diseases of approximately 75% for specialist physicians, whereas for non-specialist physicians said accuracy is reduced to 50% in diseases such as otitis media. This is particularly important considering that the consultation related to an otolaryngology disease is one of the most frequent at a primary level, especially in children.

As a result, the need to introduce a new diagnostic tool for assisting the physician during the diagnostic procedure and with which the diagnostic accuracy improves becomes apparent. In this sense, an option that has been developed successfully in other medical areas are the computer-aided diagnosis systems that through image interpretation allows the physician to obtain a second opinion about a disease.

In the state of the art are known solutions that allow assisting in the diagnosis of otolaryngology diseases, although in particular systems and methods have been proposed for the diagnosis of pathologies of middle ear only, excluding diseases related to nose and mouth.

The document U.S. Pat. No. 9,445,713 describes an apparatus and a method for the acquisition and analysis of images of the tympanic membrane. This document describes the method for assisting in the acquisition of images, as well as the identification of the region of interest (tympanic membrane), which is performed by extracting characteristics (e.g. color, texture, shape, etc.) of the acquired image. The method for the diagnosis comprises the comparison of the acquired image with each of the images of a provided database and the selection of the most similar image—the distance between its characteristics is measured—the diagnosis of the acquired image corresponds to the category of the selected image. In this document, detection tasks of inner structures of the ear are not performed, nor does the method contemplate that the identification of potential diseases considers each of said detected structures. In addition, the method described in this document is limited to identifying ear diseases from a single image and, specifically, is limited to distinguishing between otitis media with effusion, acute otitis media and normal ear.

Therefore, a single system is required to assist both specialist and non-specialist professionals to identify ear, nose, and mouth diseases and that allows to overcome deficiencies identified in the state of the art.

### SUMMARY OF THE INVENTION

The present invention provides a system for the assistance in the diagnosis of diseases from otolaryngology images of an area under examination that is characterized for comprising: an apparatus for the acquisition of images of otolaryngologic endoscopy; a processor operatively connected to said apparatus for the acquisition of images of otolaryngologic endoscopy; and a user interface comprising a screen, said user interface operatively connected to said processor;
wherein said processor is configured to recognize a type of otolaryngologic endoscopic examination apparatus to which said apparatus belongs; to obtain a plurality of otolaryngologic endoscopy images from said apparatus; to display said plurality of images on said screen and to identify, from said plurality of images, whether the same corresponds to any disease or a healthy patient;
wherein to identify from said plurality of images if the same corresponds to any disease or a healthy patient, said processor executes the tasks of determining—for each image of said plurality—if said image is focused or out of focus; detecting—in each image of said plurality considered focused—one or more inner structures of said area under examination by means of a convolutional neural network trained with a plurality of images corresponding to the type of otolaryngologic endoscopic examination apparatus to which said apparatus belongs; classifying said plurality of images using a machine learning algorithm previously trained with a plurality of data corresponding to the type of otolaryngologic endoscopic examination apparatus to which said apparatus belongs, said data that have been labeled by one or more otolaryngology professionals; and displaying on said screen of said user interface a plurality of images highlighting said one or more inner structures and one or more results of said classification;
wherein said one or more results of said classification are obtained from those classifications that may be associated with all the structures detected in those images considered focused.

In a preferred embodiment, the system is characterized in that to determine if each image of said plurality is focused or out of focus, said processor executes the Laplacian variance method.

In another preferred embodiment, the system is characterized in that said processor is additionally configured for detecting a region of interest in each of said images considered focused and in that said detection of said region of interest is executed prior to said detection of one or more inner structures. In a more preferred embodiment, the system is characterized in that for said detection of said region of interest, said processor is configured to obtain a Hough transform of each of said images considered focused.

In a further preferred embodiment, the system is characterized in that for the detection of said one or more inner structures, said processor is configured to obtain one or more characteristics from each of said images considered focused. In a more preferred embodiment, the system of the claim is characterized in that said one or more characteristics are selected from the group formed by the color, shape, texture, edges as well as combinations thereof.

In another preferred embodiment, the system is characterized in that for said detection of said one or more inner structures, said processor is configured to use a convolutional neural network that is selected from the group formed by Mask-CNN and U-Net.

In a further preferred embodiment, the system is characterized in that to perform said classification, said processor is configured to execute an algorithm that is selected from the group formed by support vectors, decision trees, nearest neighbors and deep learning algorithms.

In addition, the present invention provides an ex vivo method for assisting in the diagnosis of diseases from otolaryngology images of an area under examination characterized by comprising the steps of: providing a system that comprises: an apparatus for the acquisition of images of otolaryngologic endoscopy; a processor, operatively connected to said apparatus for the acquisition of images of otolaryngologic endoscopy; and a user interface comprising a screen, said user interface operatively connected to said processor; recognizing by means of said processor, a type of otolaryngologic endoscopic examination apparatus to which said apparatus belongs; acquiring by said processor, a plurality of images of otolaryngologic endoscopy from said apparatus; displaying said plurality of images on said screen; and identifying from said plurality of images whether the same corresponds to any disease or to a healthy patient, by means of said processor;
wherein to identify from said plurality of images if the same corresponds to any disease or to a healthy patient, said processor executes the tasks of: determining—for each image of said plurality—if said image is focused or out of focus; detecting—in each image of said plurality considered focused—one or more inner structures of said area under examination by means of a convolutional neural network trained with a plurality of images corresponding to the type of otolaryngologic endoscopic examination apparatus to which said apparatus belongs; classifying said plurality of images using a machine learning algorithm previously trained with a plurality of data corresponding to the type of otolaryngologic endoscopic examination apparatus to which said apparatus belongs, said data that have been labeled by one or more otolaryngology professionals; and displaying on said screen of said user interface a plurality of images highlighting said one or more inner structures and one or more results of said classification;
wherein said one or more results of said classification are obtained from those classifications that may be associated with all the structures detected in those images considered focused.

In a preferred embodiment, the method is characterized in that said task of detecting if each image of said plurality is focused or out of focus is performed by the Laplacian variance method.

In another preferred embodiment, the method is characterized in that it additionally comprises detecting a region of interest in each of said images considered focused by said processor; and in that said detection of said region of interest is executed prior to said detection of one or more inner structures. In a more preferred embodiment, the method is characterized in that for said step of detecting said region of interest, it comprises obtaining—by means of said processor—a Hough transform of each of said images considered focused.

In a further preferred embodiment, the method is characterized in that said step of detecting said one or more inner structures, it comprises obtaining—by means of said processor—one or more characteristics from each of said images considered focused. In a more preferred embodiment, the method is characterized in that said one or more characteristics are selected from the group formed by the color, shape, texture, edges as well as the combination thereof.

In another preferred embodiment, the method is characterized in that said step of detecting said one or more inner structure comprises using—by means of said processor—a convolutional neural network that is selected from the group formed by Mask-CNN and U-Net.

In a further preferred embodiment, the method is characterized in that said step of classifying said plurality of images comprises executing—by means of said processor—an algorithm that is selected from the group formed by support vectors, decision trees, nearest neighbors and deep learning algorithms.

### BRIEF DESCRIPTION OF THE FIGURES

FIG. 1 shows a schematic view of a first embodiment of the system which is the object of the present invention.

FIG. 2 shows a flow chart of a first embodiment of the method which is the object of the present invention.

FIG. 3 shows a flow chart of a second embodiment of the method which is the object of the present invention.

FIG. 4A shows a representative image obtained with the apparatus that is part of the system which is the object of the present invention. FIG. 4B shows an image obtained from the image illustrated in FIG. 4A, wherein the region of interest has been cut and centered.

FIG. 5A shows a representative image obtained with the apparatus that is part of the system, which is the object of the present invention, after detecting the region of interest. FIG. 5B shows an image wherein the inner structures present in the image in FIG. 5A have been recognized.

### DETAILED DESCRIPTION OF THE INVENTION

Hereinafter, the invention will be described in detail, with reference to the figures that accompany the present application.

In a first object of invention, the present invention provides a system (1) for assisting in the diagnosis of diseases from otolaryngology images of an area under examination that essentially comprises:
an apparatus (11) for the acquisition of otolaryngologic endoscopy images;
a processor (12), operatively connected to said apparatus (11) for the acquisition of otolaryngologic endoscopy images; and
a user interface (13) that comprises a screen (14), said user interface (13) operatively connected to said processor (12);
wherein said processor (12) is configured to:
recognize (21) a type of otolaryngologic endoscopic examination apparatus to which said apparatus belongs (11);
obtain (22) a plurality of images of otolaryngologic endoscopy from said apparatus (11);
display (28) said plurality of images on said screen (14); and
identify (30) from said plurality of images whether the same corresponds to any disease or to a healthy patient;
wherein to identify (30), from said plurality of images, whether the same corresponds to any disease or to a healthy patient, said processor (11) executes the tasks of:
determining (23) for each image of said plurality, if said image is focused or out of focus;
detecting (26) in each image of said plurality considered focused, one or more inner structures of said area under examination, by means of a convolutional neural network (32) trained with a plurality of images corresponding to the type of otolaryngologic endoscopic examination apparatus to which said apparatus belongs;
classifying (29) said plurality of images using a machine learning algorithm (33) previously trained with a plurality of data corresponding to the type of otolaryngologic endoscopic examination apparatus to which said apparatus belongs, said data that have been labeled by one or more otolaryngology professionals; and
displaying (28) on said screen of said user interface a plurality of images highlighting said one or more inner structures and one or more results of said classification;
wherein said one or more results of said classification are obtained from those classifications that may be associated with all the structures detected in those images considered focused.

Regarding said apparatus (11), the same may be any apparatus that allows the acquisition of otolaryngologic endoscopy images, without this limiting the scope of the present invention. Specifically, said apparatus (11) may allow the acquisition of ear, nose, mouth, or throat images, including both pharynx and larynx. For example, and without this limiting the scope of the present invention, said apparatus (11) may be selected from the group formed by otoscope, otoendoscope, nasofibroscope, laryngoscope, naso-pharyngo-laryngoscope.

Said apparatus (11) is operatively connected to said processor (12), in a manner that said processor (12) may acquire (22) a plurality of otolaryngologic endoscopy images from said apparatus (11). In a preferred embodiment, without this limiting the scope of the present invention, said processor (12) may also be configured to control said apparatus (11). For example, and without this limiting the scope of the present invention, said processor (12) may control acquisition parameters of said apparatus (11), such us, without limitation to these, acquisition frequency, exposure time, lens aperture or illumination intensity of said apparatus (11).

On the other hand, the operative connection between said apparatus (11) and said processor (12) may be obtained in a wired or wireless manner as well as a combination thereof, without this limiting the scope of the present invention. Examples of wired connections, without this limiting the scope of the present invention, are connections through USB cables, optical fiber, coaxial cables, UTP cables, STP cables, RS-232 cable, HDMI cable, among others. Moreover, examples of wireless connections, without this limiting the scope of the present invention, may be obtained by Bluetooth, Wi-Fi, pulsed laser, among others.

Regarding said user interface (13), the same comprises a screen (14) and may comprise input devices for the interaction with a user of the system which is object of the present invention. For example, and without this limiting the scope of the present invention, said input devices may be selected from the group formed by keyboards, microphones, touch screens, mouse, cameras, as well as the combination thereof. In a preferred embodiment, said screen (14) is a touch screen. In addition, said user interface (13) may comprise additional output devices to said screen (14). For example, and without this limiting the scope of the present invention, said output devices may be selected from the group formed by speakers, lights, screens, as well as the combination thereof.

On the other hand, in the context of the present invention, it must be understood that said user interface (13) is operatively connected to said processor (12) when said processor can control said user interface (13) to display images on said screen (14). Additionally, in those preferred embodiments in which said user interface (13) comprises input devices, said processor (12) may be configured to obtain information corresponding to the interaction with a user from said input devices. In other preferred embodiments, in which said user interface comprises additional output devices to said screen (14), said processor (12) may be configured to control said additional output devices.

Said processor (12) is also configured to recognize (21) a type of otolaryngologic endoscopic examination apparatus to which said apparatus (11) belongs. Said recognition (21) may be obtained automatically or manually without this limiting the scope of the present invention. For example, and without this limiting the scope of the present invention, a user of the system (1) which is object of the present invention, may select a type of otolaryngologic endoscopic examination apparatus to which said apparatus (11) belongs through said user interface (13). For this purpose, for example and without this limiting the scope of the present invention, said processor (12) may be configured to display a list of types of otolaryngologic endoscopic examination apparatus on the screen (14) of said user interface (13). Nevertheless, in another preferred embodiment, said recognition (21) may be performed automatically. For example, and without limiting the scope of the present invention, said processor (12) may be configured to obtain an identifier from said apparatus (11) and to search said identifier in a classified list of identifiers of types of otolaryngologic endoscopic examination apparatus. Said identifier may be, for example and without limiting to these, a MAC address, or a static IP address. In other preferred embodiments, without limiting the scope of the present invention, said apparatus (11) can incorporate information corresponding to its brand, model, and/or serial number, as metadata of one or more images obtained. In this case, said processor (12) may be configured to obtain said identifier from said metadata.

In addition, and as previously mentioned, said processor (12) is configured to obtain (22) a plurality of otolaryngologic endoscopy images from said apparatus (11). As previously mentioned, said obtainment of said plurality of images may be carried out by wired or wireless means, without this limiting the scope of the present invention. Additionally, said plurality of images may correspond to a plurality of photographs acquired by said apparatus (11), to a video formed by a plurality of frames or to a combination of both, without this limiting the scope of the present invention. The number of images that are part of said plurality does not limit the scope of the present invention, provided it is greater than or equal to 2. In a preferred embodiment, without this limiting the scope of the present invention, said plurality of images comprises between 2 and 100,000 images, more preferably between 15,000 and 80,000 images and even more preferably 40,000 images.

If said plurality of images corresponds to a video, the length of said video does not limit the scope of the present invention. For example, and without this limiting the scope of the present invention, said video may have a length of between 1 minute and 30 minutes, more preferably between 5 and 15 minutes. The frame rate at which said video is obtained does not limits the scope of the present invention either. For example, and without this limiting the scope of the present invention, said frame rate may be between 10 and 100 frames per second (FPS), more preferably between 20 and 50 FPS and even more preferably 30 FPS. In those preferred embodiments in which said processor (12) is configured to control said apparatus (11), for example and without this limiting the scope of the present invention, said processor (12) may be configured to control said frame rate.

The obtainment (22) of said plurality of images may be substantially carried out in real time, while the otolaryngologic endoscopic examination is being performed, or after the acquisition of said plurality of images by means of said apparatus (11) without this limiting the scope of the present invention. In the context of the present invention, a situation in which the time difference between the acquisition of the images by means of the apparatus (11) and their obtainment by means of the processor (12) is less than a certain threshold time and it must be understood as substantially in real time. For example, and without limiting the scope of the present invention, said threshold time may be less than 1 second, more preferably less than 500 milliseconds and even more preferably less than 100 milliseconds. On the other hand, in the context of the present invention, it will be understood that said obtainment (22) is subsequent to said acquisition when it is performed in a time longer than said threshold time.

In addition, said processor (12) is configured to display (28) said plurality of images on said screen (14). Just like the obtainment (22) of said plurality of images, said display (28) may be substantially performed in real time or subsequently to the obtainment (22) of said plurality of images, without limiting the scope of the present invention.

Said processor (12) is configured to identify (30) from said plurality of images, whether the same corresponds to any disease or to a healthy patient. To perform said identification (30) said processor (12) executes a series of tasks from said plurality of images.

Firstly, for each image of said plurality, said processor (12) is configured to determine (23) if said image is focused or out of focus. This presents at least two significant advantages, without limiting the scope of the present invention. On the one hand, by avoiding analyzing images out of focus, said plurality of images are avoided from being classified in a wrong way. On the other hand, by avoiding analyzing out of focus images, the computational power required for the image analysis is reduced.

Said processor (12) may determine (23), for each image of said plurality, if said image is focused or out of focus by means of any method known to a person normally skilled in the art. For example, and without this limiting the scope of the present invention, said determination (23) may be performed by a method chosen from the group formed by methods based on variance of Laplacian filter, Gaussian filter, Canny's algorithm, Sobel operator, thresholding methods, phase detection and contrast detection, methods based on wavelet, methods based on gradients, as well as the combinations thereof. In a preferred embodiment, without this limiting the scope of the present invention, to determine (23) if each one of said images of said plurality is focused or out of focus, said processor (12) executes the Laplacian variance method.

Subsequently to said step, said processor (12) is configured to detect (26) in each image of said plurality considered focused, one or more inner structures of said area under examination. For this, said processor (12) uses a convolutional neural network (32) trained with a plurality of images corresponding to the type of otolaryngologic endoscopic examination apparatus to which said apparatus belongs (11). The nature of said convolutional neural network (32) as well as the number of images that have been used to train said convolutional neural network (32) does not limit the scope of the present invention. In the context of the present invention, it must be understood that for each type of otolaryngologic endoscopic examination apparatus said processor (12) is configured to use a corresponding convolutional neural network (32) trained with a plurality of images corresponding to said type of apparatus. For example, and without limiting the scope of the present invention, said processor (12) may use a convolutional neural network (32) trained with ear images when said apparatus (11) is an otoscope or otoendoscope. In another embodiment example, without limiting the scope of the present invention, said processor (12) may use a convolutional neural network (32) trained with images of nostrils when said apparatus (11) is a nasolaryngoscope or nasofibroscope.

The method whereby said processor (12) detects (26) said one or more inner structures does not limit the scope of the present invention and any method known to a person normally skilled in the art may be used. In a preferred embodiment, without limiting the scope of the present invention, said processor (12) may be configured to obtain one or more characteristics from each of the images considered focused. In the context of the present invention, the relevant information obtained from an image or a part thereof, both at a pixel level and of a set of pixels must be understood as a characteristic. For example, and without this limiting the scope of the present invention, said one or more characteristics may be selected from the group formed by the color, shape, texture, edges, as well as the combination thereof. In this sense, for example and without limiting the scope of the present invention, said convolutional neural network (32) may determine the presence of one or more inner structures in one of the images considered focused by applying a learned model that uses said one or more characteristics. In addition, said convolutional neural network (32) may be trained, for example and without limiting the scope of the present invention, to determine the likelihood that an individual pixel of said image considered focused corresponds to any of the inner structures of the area under examination.

The nature of said convolutional neural network (32) does not limit the scope of the present invention and any convolutional neural network (32) known to a person normally skilled in the art may be used. In a preferred embodiment, said processor (12) is configured to use a convolutional neural network (32) that is selected from the group formed by Mask-CNN, U-Net that are used for semantic segmentation tasks and VGG-17, ResNet-50, Inception V3 that are used for classification and detection tasks as well as a combination of them. Additionally, for different types of apparatus, said processor (12) may use different types of convolutional neural networks (32) without limiting the scope of the present invention.

In some preferred embodiments, without this limiting the scope of the present invention, said processor may be configured to detect (24) in each of the images considered focused, a region of interest (ROI) and to crop said images considered focused around said region of interest. For example, and without limiting the scope of the present invention, in FIG. 4A is illustrated an image obtained by said processor (12) from said apparatus (11), wherein the region of interest has been highlighted for illustrative purposes. On the other hand, FIG. 4B illustrates an image generated by said processor (1) wherein the image has been centered and cropped to substantially maintain only the detected region of interest. In a preferred embodiment, said detection (24) is performed prior to the step of detecting (26) the inner structures of the area under examination. On the one hand, the above mentioned allows to reduce the computational power required for image analysis. On the other hand, it allows that by displaying the analyzed images on the screen (14) of the user interface (13) all the images have substantially the same size. Any method known in the state of the art may be used to detect (24) said region of interest without limiting the scope of the present invention. For example, said detection (24) of said region of interest may be performed by a method chosen from the group formed by Sobel operator, Canny's algorithm, thresholding methods, local color descriptors, color consistency vectors, histogram of gradients, grid color moment, as well as the combination of them. In a preferred embodiment, without limiting the scope of the present invention, to obtain said detection (24) of said region of interest, said processor (12) is configured to obtain a Hough transform of each of said images considered focused.

Subsequently to said detection (26) of said one or more inner structures, said processor (12) is configured to classify (29) said plurality of images using a machine learning algorithm (33). Said machine learning algorithm (33) has previously been trained with a plurality of data corresponding to the type of otolaryngologic endoscopic examination apparatus to which said apparatus belongs. Additionally, said data have been labeled by one or more otolaryngology professionals. Just as for the convolutional neural network (32), the nature of said machine learning algorithm (33), as well as the size of the data set that has been used to train said machine learning algorithm (33) does not limit the scope of the present invention. In the context of the present invention, it must be understood that for each type of otolaryngologic endoscopic examination apparatus, said processor (12) is configured to use a corresponding machine learning algorithm (33), trained with data corresponding to said type of apparatus. For example, and without this limiting the scope of the present invention, said processor (12) may use a machine learning algorithm trained with ear data when said apparatus (11) is an otoscope or otoendoscope. In another embodiment example, without limiting the scope of the present invention, said processor (12) may use a machine learning algorithm (33) trained with data of nostrils when said apparatus (11) is a nasolaryngoscope or nasofibroscope.

The machine learning algorithm (33) whereby said processor (12) performs said classification (29) does not limit the scope of the present invention and any algorithm known to a person normally skilled in the art may be used. In a preferred embodiment, without limiting the scope of the present invention, said processor (12) may be configured to execute a machine learning algorithm (33) that is selected from the group formed by support vectors, decision trees, nearest neighbors and deep learning algorithms. Additionally, for different types of apparatus, said processor (12) may execute different types of machine learning algorithms (33) without this limiting the scope of the present invention.

Said classification (29) considers all the structures that have been detected (26) from said plurality of images. This has an advantage in comparison with the state of the art, wherein the classification is performed based on individual images, in which all the relevant inner structures may not be present. Therefore, the system (1) which is object of the present invention, obtains said results of said classification (29) from those classifications that may be associated with all the structures detected in those images considered focused. For example, and without this limiting the scope of the present invention, said result of classification (29) may assign a probability value to said plurality of images for each of the diseases that said machine learning algorithm (33) allows to identify. Besides, said probability value may be updated to the extent that said plurality of images is obtained (22), without this limiting the scope of the present invention. For example, and without limiting the scope of the present invention, the foregoing may be carried out when said obtainment (22) is performed substantially in real time.

Said machine learning algorithm (33) allows in an advantageous manner, the assistance in the diagnosis of a high number of diseases of the ear, nose, and mouth. For example, and without limiting the scope of the present invention, when the apparatus (11) corresponds to an apparatus for the acquisition of ear images, said machine learning algorithm (33) may be trained with a data set corresponding to a plurality of ear diseases that may include, but are not limited to, ear wax, otitis externa, otitis media with effusion, acute otitis media, chronic otitis media, tympanic retraction, foreign body, exostoses of the auditory canal, osteoid osteomas, mono-and dimeric, myringosclerosis, eardrum perforation and normal condition.

On the other hand, without limiting the scope of the present invention, when the apparatus (11) corresponds to an apparatus for the acquisition of nose images, said machine learning algorithm (33) may be trained with a set of data corresponding to a plurality of nose diseases that may include, but are not limited to, nostril diseases such as normal nostril, blood in nostril, mucous rhinorrhea, purulent rhinorrhea, tumors, polyps; diseases in nasal septum, such as normal nasal septum, deviated septum, altered mucosa, dilated vessels, bleeding points, scabs, ulcer, and diseases in inferior turbinate such as normal inferior turbinate, turbinate hypertrophy, polyps.

In another preferred embodiment, without this limiting the scope of the present invention, when the apparatus (11) corresponds to an apparatus for the acquisition of mouth images, said machine learning algorithm (33) may be trained with a data set corresponding to a plurality of mouth diseases that may include, but are not limited to palate diseases such as normal palate, bifid uvula, uvula papilloma, palate edema, ulcers; oropharyngeal diseases, such as normal oropharynx, pharyngeal cobblestoning, ulcers, and tongue diseases such as normal tongue, glossitis, ulcer, erythroplakia.

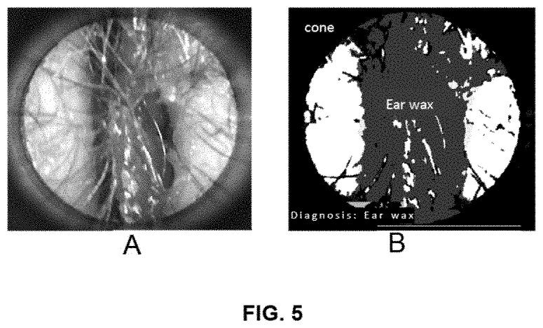

In addition, said processor (12) is configured to display (28) on said screen (14) of said user interface (13) a plurality of images highlighting said one or more inner structures and one or more results of said classification (29). For example, and without limiting the scope of the present invention, FIG. 5A illustrates a representative image wherein the inner structures of the ear are observed. On the other hand, FIG. 5B illustrates an image where said inner structures have been detected and highlighted. In addition, the diagnosis resulting from the classification (29) of said image has been incorporated in said image illustrated in FIG. 5B In those preferred embodiments in which said processor (12) assigns a probability value to said plurality of images for each of the diseases that said machine learning algorithm (33) allows to identify, said display (28) may include all those diseases that exceed a certain probability threshold value as well as their corresponding probability value. In this latter preferred embodiment, without limiting the scope of the present invention, said probability threshold value can take any value that allows an adequate assistance in the diagnosis. For example, and without this limiting the scope of the present invention, said threshold value may be greater than a probability value of 0.5; more preferably greater than 0.7; and even more preferably greater than 0.9. In addition, in those preferred embodiments in which the user interface (13) comprises input devices and without this limiting the scope of the present invention, said processor (12) may be configured to receive a probability threshold value by means of said input devices.

In this way, it is possible to provide a system (1) for the assistance in the diagnosis of diseases from otolaryngology images of an area under examination that allows to overcome the deficiencies of the state of the art. It must be understood that all of the options described for different technical characteristics can be combined with each other or with other options known to a person normally skilled in the art, in any expected manner, without this limiting the scope of the present invention.

In addition, the present invention provides an ex vivo method (2) for the assistance in the diagnosis of diseases from otolaryngology images that essentially comprises the steps of:
providing a system (1) that comprises: an apparatus (11) for the acquisition of otolaryngologic endoscopy images; a processor (12), operatively connected to said apparatus (11) for the acquisition of otolaryngologic endoscopy images; and a user interface (13) comprising a screen (14), said user interface (13) operatively connected to said processor (11);
   recognizing (21) by means of said processor (12) a type of otolaryngologic endoscopic examination apparatus to which said apparatus belongs (11);
   acquiring (22) by means of said processor (12), a plurality of otolaryngologic endoscopy images from said apparatus (11);
   displaying (28) said plurality of images on said screen (14); and
   identifying from said plurality of images, if it corresponds to any disease or to a healthy patient, by means of said processor (12);
   wherein to identify—from said plurality of images—if the same corresponds to any disease or to a healthy patient, said processor executes the tasks of:
   detecting (23) for each image of said plurality, if said image is focused or out of focus;
   detecting (26) in each image of said plurality considered focused, one or more inner structures of said area under examination by means of a convolutional neural network (32) trained with a plurality of images corresponding to the type of otolaryngologic endoscopic examination apparatus to which said apparatus belongs (11);
   classifying (29) said plurality of images using a machine learning algorithm (33) previously trained with a plurality of data corresponding to the type of otolaryngologic endoscopic examination apparatus to which said apparatus (11) belongs, said data that have been labeled by one or more otolaryngology professionals; and
   displaying (28) on said screen (14) of said user interface (13) a plurality of images highlighting said one or more inner structures and one or more results of said classification (29);
   wherein said one or more results of said classification are obtained from those classifications that may be associated with all the structures detected in those images considered focused.

All those options previously described for the system (1) which is object of the present invention may be applied to the ex vivo method (2) which is object of the present invention, without limiting the scope of the present invention. Particularly, without limiting the scope of the present invention, all the options described for the tasks that performed by said processor (12) may be applied to the ex vivo method (2) which is object of the present invention.

Additionally, it must be understood that all of the options described for different technical characteristics may be combined with each other or with other options known to a person normally skilled in the art, in any expected manner, without limiting the scope of the present invention.

The system (1) and method (2) that are object of the present invention allow—advantageously and without limiting the scope of the present invention—to encompass a number of ear, nose, and/or mouth diseases that is much greater than the solutions known in the state of the art. In addition, due to its modular nature, the system (1) and method (2) that are object of the present invention are perfectly scalable to include more diseases as required by a user of the system (1) and/or method (2) that are object of the present invention. Hereinafter, examples of embodiment of the present invention will be described. It must be understood that said examples are given in order to provide a better understanding of the invention, however, under any circumstances limit the scope of the protection sought. Furthermore, options of technical characteristics described in different examples may be combined with each other or with options previously described in this descriptive memory, in any manner expected by a person normally skilled in the art without this limiting the scope of the present invention.

### EXAMPLE 1: FIRST EMBODIMENT OF THE EX VIVO METHOD FOR THE ASSISTANCE IN THE DIAGNOSIS OF DISEASES

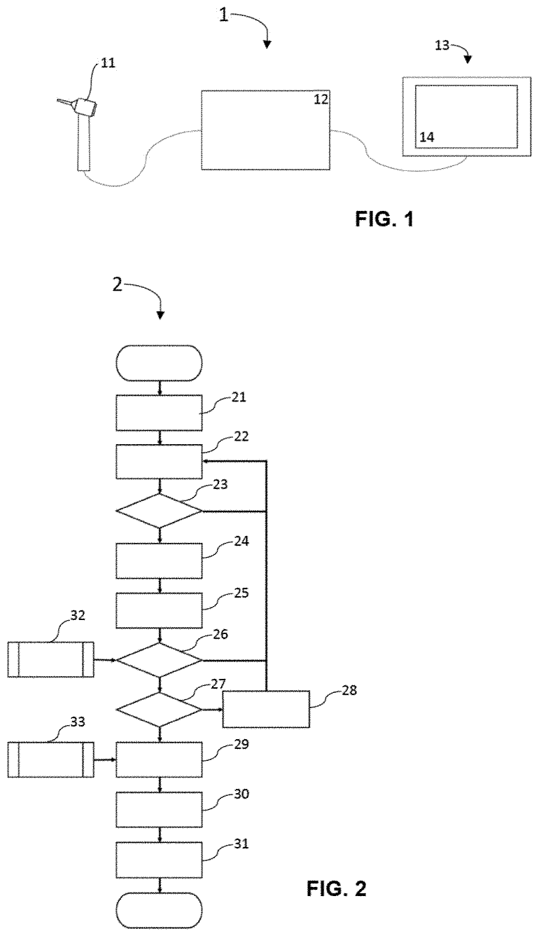

FIG. 2 illustrates a flow chart of a first embodiment of the ex vivo method (2) which is object of the present invention.

In a first stage, the processor (12) recognizes (21) the type of otolaryngologic endoscopic examination apparatus to which the apparatus (11) belongs that is part of the system (1) which is object of the present invention.

After having recognized (21) said type of apparatus, the processor (12) obtains (22) a plurality of images from said apparatus. In this embodiment of the ex vivo method (2), the processor (12) is configured to determine (23)—for each image of said plurality—if the same is focused or out of focus. If it is out of focus, said processor (12) obtains (22) the next image of said plurality. If it is focused, said processor (12) is configured to detect (24) a region of interest (ROI) in said image considered focused.

Once that said region of interest (ROI) has been detected (24), said processor (12) is configured to crop (25) said image considered focused, substantially maintaining only said region of interest (ROI).

Said processor (12) is configured to use a convolutional neural network (32) to detect (26) in said cropped image, one or more inner structures. If said image did not contain inner structures or if the same could not be properly recognized, said processor (12) obtains (22) the next image of said plurality. If said image had at least one inner structure, said processor (12) is configured to determine (27) if the examination has finished. If said examination has not finished, said processor (12) displays (28) said image on the screen (14) of the user interface (13), highlighting the identified inner structures and obtaining (22) the next image of said plurality.

If the examination has finished, said processor (12) is configured to classify (29) said plurality of images, using for this a machine learning algorithm (33). Said classification (29) allows to perform a diagnosis (30) which is finally reported (31) to the user of the system (1) which is object of the present invention.

### EXAMPLE 2: SECOND EMBODIMENT OF THE EX VIVO METHOD FOR THE ASSISTANCE IN THE DIAGNOSIS OF DISEASES

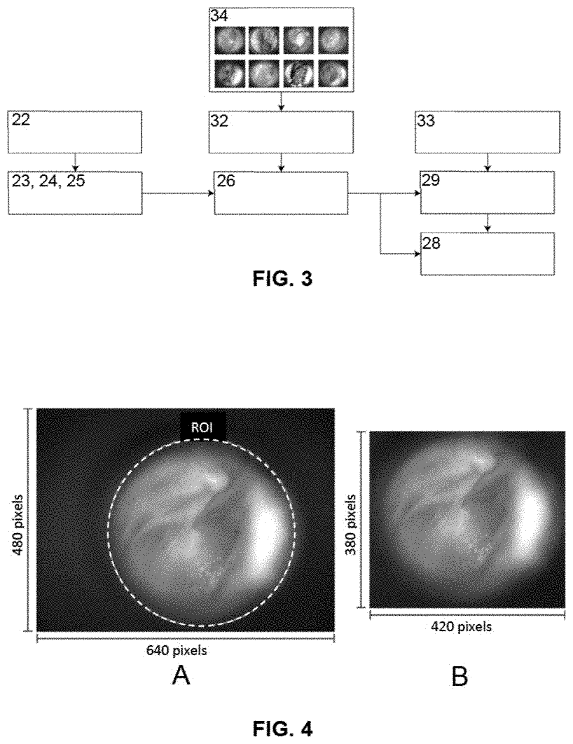

FIG. 3 shows a flow chart of a second embodiment to the ex vivo method which is object of the present invention.

In a first stage, after recognition (21) of the type of otolaryngologic endoscopic examination apparatus to which the apparatus (11) that is part of the system (1), object of the present invention belongs, said processor (12) obtains (22) a plurality of images that form a video from said apparatus (11).

Each of the frames of said video is pre-processed by said processor (12). For this purpose, said processor (12) determines (23), for each frame, if it is focused or out of focus, detects (24) a region of interest and crops (25) said frame around said region of interest. Subsequently, said processor (12) detects (26), for each pre-processed frame, one or more inner structures of the area under examination, using a convolutional neural network model (32) previously trained with a database (34) containing images of said area under examination. Once said one or more structures have been detected (26), said plurality of images, on the one hand, are displayed (28) on the screen (14) of the user interface, and on the other, they are added to a machine learning model (33) for classification (29). Said result of said classification (29) is also displayed (28) on said screen.

1. A system for assisting with the diagnosis of diseases from otolaryngology images of an area under examination, CHARACTERIZED in that comprises:
an apparatus for the acquisition of otolaryngologic endoscopy images;
a processor, operatively connected to said apparatus for the acquisition of otolaryngologic endoscopy images; and
a user interface that comprises a screen, said user interface operatively connected to said processor;
wherein said processor is configured to:
recognize a type of otolaryngologic endoscopic examination apparatus to which said apparatus belongs;
obtain a plurality of images of otolaryngologic endoscopy from said apparatus;
display said plurality of images on said screen; and
identify from said plurality of images, whether the same corresponds to any disease or to a healthy patient;
wherein to identify from said plurality of images, whether the same corresponds to any disease or to a healthy patient, said processor executes the tasks of:
determining, for each image of said plurality, if said image is focused or out of focus;
detecting in each image of said plurality considered focused, one or more inner structures of said area under examination, by means of a convolutional neural network trained with a plurality of images corresponding to the type of otolaryngologic endoscopic examination apparatus to which said apparatus belongs;
classifying said plurality of images using a machine learning algorithm previously trained with a plurality of data corresponding to the type of otolaryngologic endoscopic examination apparatus to which said apparatus belongs, said data that have been labeled by one or more otolaryngology professionals; and
displaying on said screen of said user interface a plurality of images highlighting said one or more inner structures and one or more results of said classification;
wherein said one or more results of said classification are obtained from those classifications that may be associated with all the structures detected in those images considered focused.

2. The system of claim 1, CHARACTERIZED in that to detect if each image of said plurality is focused or out of focus, said processor executes the Laplacian variance method.

3. The system of claim 1, CHARACTERIZED in that said processor, additionally, is configured to detect a region of interest in each one of said images considered focused and in that said detection of said region of interest is executed prior to said detection of one or more inner structures.

4. The system of claim 3, CHARACTERIZED in that for said detection of said region of interest, said processor is configured to obtain a Hough transform of each of said images considered focused.

5. The system of claim 1, CHARACTERIZED in that for the detection of said one or more inner structures, said processor is configured to obtain one or more characteristics from each of said images considered focused. The system of claim 5, CHARACTERIZED in that said one or more characteristics are selected from the group formed by the color, shape, texture, edges as well as the combinations thereof.

7. The system of claim 1, CHARACTERIZED in that for said detection of said one or more inner structures, said processor is configured to use a convolutional neural network that is selected from the group formed by Mask-CNN and U-Net.

8. The system of claim 1, CHARACTERIZED in that to perform said classification, said processor is configured to execute an algorithm that is selected from the group formed by support vectors, decision trees, nearest neighbors, and deep learning algorithms.

9. An ex vivo method for the assistance in the diagnosis of diseases from otolaryngology images of an area under examination, CHARACTERIZED in that comprises the steps of:
providing a system that comprises: an apparatus for the acquisition of images of otolaryngologic endoscopy; a processor, operatively connected to said apparatus for the acquisition of images of otolaryngologic endoscopy; and a user interface comprising a screen, said user interface operatively connected to said processor;
recognizing, by means of said processor, a type of otolaryngologic endoscopic examination apparatus to which said apparatus belongs;
obtaining, by means of said processor, a plurality of images of otolaryngologic endoscopy from said apparatus;
displaying said plurality of images on said screen; and
identifying from said plurality of images, whether the same corresponds to any disease or to a healthy patient, by means of said processor;
wherein to identify from said plurality of images, whether the same corresponds to any disease or to a healthy patient, said processor executes the tasks of:
determining, for each image of said plurality, if said image is focused or out of focus;
detecting in each image of said plurality considered focused, one or more inner structures of said area under examination, by means of a convolutional neural network trained with a plurality of images corresponding to the type of otolaryngologic endoscopic examination apparatus to which said apparatus belongs;
classifying said plurality of images using a machine learning algorithm previously trained with a plurality of data corresponding to the type of otolaryngologic endoscopic examination apparatus to which said apparatus belongs, said data that have been labeled by one or more otolaryngology professionals; and
displaying on said screen of said user interface a plurality of images highlighting said one or more inner structures and one or more results of said classification;
wherein said one or more results of said classification are obtained from those classifications that may be associated with all the structures detected in those images considered focused.

10. The method of claim 9, CHARACTERIZED in that said task of detecting if each image of said plurality is focused or out of focus is performed by means of the Laplacian variance method.

11. The method of claim 9, CHARACTERIZED in that it additionally comprises detecting a region of interest in each one of said images considered focused by means of said processor; and in that said detection of said region of interest is executed prior to said detection of one or more inner structures.

12. The method of claim 11, CHARACTERIZED in that for said step of detecting said region of interest, it comprises obtaining by means of said processor, a Hough transform of each of said images considered focused.

13. The method of claim 9, CHARACTERIZED in that said step of detecting said one or more inner structures comprises obtaining, by means of said processor, one or more characteristics from each of said images considered focused.

14. The method of claim 13, CHARACTERIZED in that said one or more characteristics are selected from the group formed by the color, shape, texture, edges as well as the combinations thereof.

15. The method of claim 9, CHARACTERIZED in that said step of detecting said one or more inner structures, comprises using, by means of said processor, a convolutional neural network that is selected from the group formed by Mask-CNN and U-Net.

16. The method of claim 9, CHARACTERIZED in that said step of classifying said plurality of images comprises executing, by means of said processor, an algorithm that is selected from the group formed by support vectors, decision trees, nearest neighbors and deep learning algorithms.
